(19) United States
(12) Patent Application Publication
(10) Pub. No.: US 2008/0299160 A1
(43) Pub. Date: Dec. 4, 2008

(54) Method of Manufacture of Polymer Composites

(76) Inventors: Ochayi C. Agboh, Hampshire (GB); Michael J. Pitkethly, Northumberland (GB)

Correspondence Address: NIXON & VANDERHYE, PC, 901 NORTH GLEBE ROAD, 11TH FLOOR, ARLINGTON, VA, 22203, US

(21) Appl. No.: 10/586,489

(22) PCT Filed: Jan. 27, 2005
(86) PCT No.: PCT/GB2005/000261

§ 371 Date: Jul. 20, 2006

(30) Foreign Application Priority Data
Jan. 28, 2004 (GB) 0401821.4

(51) Int. Cl. A61K 9/70; B29C 47/00; A61P 31/04; A61F 13/00

(52) U.S. Cl. 424/405; 264/176.1; 264/211.1; 424/618; 424/630; 424/641; 424/649; 424/651; 424/653

(57) ABSTRACT

A simple and cost effective method of producing a polymer composite comprising a polymer matrix having metal nanoparticles incorporated therein comprises the steps of (i) mixing metal nanoparticles with a polymer dope; and (ii) solidifying the polymer composite from the dope. Antimicrobial fibres are produced by extruding a dope solution (1) held in a container (2) provided with an inert atmosphere (3) into a coagulating bath (4) containing a coagulant (5), by means of a pump (6) and a spinneret head (7) completely immersed in the coagulant. The dope is filtered via a filter (8) positioned behind the spinneret.

The present invention relates to a method of manufacturing a polymer composite comprising a polymer matrix having metal nanoparticles incorporated therein, and products relating thereto. In particular, the present invention relates to anti-microbial fibres and films incorporating metal nanoparticles, and more especially to polymer/silver fibres, and fabrics and wound dressings made therefrom.

Many metals, including transition metals, have been applied to fibres, especially polymeric fibres, so as to produce textiles and other materials that exhibit beneficial properties. The metal may be applied to the fibre substrate either in its metallic form or as a salt or compound, and typical methods of application include vapour deposition, sputtering, coating, spraying, chemical reactions and the use of adhesives. Such methods tend, however, to be inefficient and costly and may result in the release of toxic metals into the environment. Furthermore, because the metal or metal compound is typically only present at the fibre surface, the required material property is not generally permanent; in other words, the property degrades with time.

One such example of a beneficial property is antimicrobial activity, and polymeric fibres incorporating antimicrobial metals are known for the production of wound dressings, surgical apparel and other antimicrobial materials. Many metals exhibit antimicrobial effects, for example Ag, Au, Pt, Pd, Ir, Sn, Cu, Sb, Bi and Zn, and their mode of action is typically due to the interference of the metal ion with electron donating functional groups present in microbiological molecules. The antimicrobial metal may be applied to the fibre in its metallic form, but more commonly it is applied as a metal compound.

Ag, in particular, has long been established as an effective anti-microbial agent and is widely used in bio-medical products to control infection. Usually a silver salt or compound, such as silver nitrate, is used as the active agent in antimicrobial fibres, particularly in applications involving the use of a hydrophilic substrate, such as wound dressings. Use of the metal itself in wound dressings and such-like is generally undesirable, because of the inherent hydrophobicity of metal coatings etc.

However, certain disadvantages are associated with the use of silver compounds, including photosensitivity, toxic effects such as hypochloremia and hyperpyexia, transient discoloration of patients skins and rapid dissipation (leaching) of the antimicrobial agent.

An object of the present invention is to provide a improved method for the production of antimicrobial materials and products deriving therefrom.

According to a first aspect of the present invention, there is provided a method of producing a polymer composite comprising a polymer matrix having metal nanoparticles incorporated therein, said method comprising the steps of:
(i) mixing metal nanoparticles; with a polymer dope; and
(ii) solidifying the polymer composite from the dope.

A polymer dope typically comprises a solvent and a polymer, and is formed by mixing those ingredients in a mixer. A polymer dope can be used as a means of solidifying the polymer into various forms, such as, for example, fibres, threads or other extruded shapes, sheets, films, membranes or cast shapes. The polymer may be reconstituted, or solidified, from the solvent by various means, for example by coagulation or by solvent evaporation. Two common examples of the use of a polymer dope are; firstly, in conjunction with a spinning technique to form threads and fibres; and, secondly, casting the dope into a film, sheet or membrane by spreading the dope onto a surface with a doctor blade.

Metal nanoparticles are metal particles having nanometric dimensions, and may have, for example, dimensions in the order of a few nanometres to several hundred nanometres. The metal nanoparticles may be spherical or aspherical, and may also be known as a metal nanopowder or as a nanometric metal. The metal nanoparticles used in the present invention may comprise a metal, a metal alloy or a metalloid, or any combination thereof.

The present inventors have realised that, by incorporating metal nanoparticles into a polymer dope prior to reconstituting the polymer from the dope, the metal and polymer can be co-solidified so as to form a composite polymeric material having metal nanoparticles incorporated therein. The method is, in effect, a physical process of dispersing a solid metal powder into a polymer solution and then binding the metal and polymer together through solidification. The composite material thus-formed comprises a continuous polymer phase, or matrix, throughout which the metal nanoparticles are embedded, and is referred to herein as a polymer composite. Polymer composites manufactured according to the present invention can be made in a simple and cost effective manner, with little wastage of expensive metal starting materials.

Furthermore, by selecting specific combinations of metal nanoparticles and polymer matrix, polymer composites having a wide range of desirable properties can be obtained.

Preferably, the polymer composite is formed by an extrusion method, in which case the polymer and metal nanoparticles are co-extruded so as to form an extruded composite material. Advantageously, the polymer composite is extruded in the form of a fibre, or thread, so that a fibre comprising a polymer matrix incorporating metal nanoparticles is obtained. Such fibres are particularly useful for making fabrics.

Alternatively, the polymer may be extruded in the form of a thin sheet or film. The present invention is of particular benefit where the extruded polymer composite has dimensions of microns or even nanometers, due to the small size of the metal particles.

By varying the concentration of metal nanoparticles mixed with the polymer dope during the extrusion process, fibres and other extruded products may be obtained having a metal concentration that varies along the extruded length.

Fibres may be extruded from the polymer dope by any suitable technique, but, preferably, polymer composite fibres are extruded by a spinning technique such as wet spinning, dry spinning or dry-jet wet spinning. In wet spinning, the dope solution is extruded through a spinneret which is completely immersed in a coagulant. In dry-jet wet-spinning a spinneret is also employed, but a small air gap is left between the spinneret face and the surface of the coagulant, the length of air gap depending on the polymer concentration and viscosity. A dry spinning technique tends to be preferred for polymers dissolved in a volatile solvent, whereas wet spinning or dry-jet wet spinning is more suitable for aqueous or non-volatile dope solutions.

Surprisingly, metal nanoparticles can be co-spun from a polymer dope without any chemical or physical modification of the metal. In other words, metal ions, complexes and so on need not be formed and the metal need not be dissolved in the polymer dope. Nor is any chemical reaction or chemical bond between the polymer and the metal required. It has been found that metal nanoparticles are physically bound into the polymer matrix by the spinning process itself, and retained securely therein. The result is a simple manufacturing process with little wastage of metal or polymer.

Preferably, the nanoparticles have a particle size less than 500 nm, more preferably less than 200 nm and even more preferably less than 100 nm. For fibre spinning applications, the particle size is preferably in the range 20 to 100 nm. When powdered nanoparticles are added directly to the polymer dope, they need not have a particle-size any smaller than 20 nm, unlike prior art methods in which very fine nanoparticles are used in conjunction with chemical dispersants. In Example 1, the particle size distribution was d90<70 nm, d50<50 nm and d10<40 nm.

The nanoparticles may be either spherical or aspherical, but in the case of fibre spinning aspherical particles, for example rods, are advantageous. Non-regular features on the particle surface, such as angles or spikes, may be desirable, for example where electrical conductivity is required.

The metal nanoparticles can be added to the solvent before preparing the polymer dope, but preferably the polymer dope is fully prepared prior to adding the metal nanoparticles. The inventors have found that it is particularly important to add the metal nanoparticles after preparing the dope in instances where the metal may interfere with the solubility of the polymer, for example where the metal concentration is relatively high. Another benefit of adding the metal nanoparticles to the prepared dope is that the concentration of nanoparticles in the dope, and thereby also in the extruded polymer composite, can be increased, decreased or otherwise controlled throughout the extrusion process. In either case, the metal nanoparticles may be added in a continuous or batchwise fashion.

Preferably, the nanoparticles are added directly to the polymer dope in the form of a powder, without first forming a dispersion, emulsion, suspension etc. Adding the metal directly to the dope as a powder results in a simple yet controllable production process and, furthermore, it has unexpectedly been found that, in the case of spun fibres, incorporation of the metal particles into the polymer is highly efficient, thereby leading to little or no wastage of expensive metal starting material.

Advantageously, the dope solution is stirred vigorously so as to produce a homogeneous polymer/nanoparticle mixture, using, for example, a high shear mixer. The benefits of forming a homogeneous mixture are, firstly, that the metal nanoparticles are uniformly dispersed across the cross-section of the extruded polymer composite and, secondly, that particle agglomeration is controlled.

If desired, a dispersant or surfactant, for example an alcohol-based dispersant, may be added to the dope solution to control agglomeration of the metal nanoparticles, such that agglomeration is reduced or even substantially prevented. Alternatively, agglomeration can be controlled by sonification or similar techniques. However, in practice it has been found that a dispersant or surfactant is rarely needed for spun metal/polymer fibres and that some degree of agglomeration is acceptable. This further simplifies the production of spun metal/polymer fibres because the dispersant or surfactant need not be removed in an additional step.

The amount of metal nanoparticles added to the dope depends upon the required application and the type of polymer selected as the matrix material. Typically, silver is added to an alginate polymer in an amount between 0.1 and 15% w/w, and to a matrix comprising cellulose in an amount up to about 5% w/w.

The polymer matrix may comprise a synthetic polymer, a natural polymer or any combination thereof. Suitable natural polymers are polysaccharides such as cellulose, alginate, chitin or chitosan. A single natural polymer may be used, or a mixture of natural polymers, for example sodium alginate mixed with carboxymethyl cellulose, pectin or xanthan.

Any synthetic polymer which is suitable for use in the chosen process can be used, such as, for example, polyethylene (PE), polyethylene terephthalate (PET), nylon, acrylic, rayon, Spandex, polyolefins, polyurethane and electromeric polymers such as Lycra. Preferably the polymer is a linear polymeric material having fibre forming characteristics. Examples of synthetic polymers that are suitable for solution spinning are polyacrylonitrile, acetate fibres or viscose fibres, but the choice of polymer is not limited to those examples.

The concentration of polymer in the dope depends on the polymer used and the application. In Example 1, 5-7% w/w alginate polymer was used to form silver/alginate fibres.

The metal nanoparticles preferably comprise a transition metal, but may comprise any other metal, metal alloy or metalloid, or any combination thereof, which exhibits the properties required in the extruded polymer composite.

Preferably, the metal nanoparticles have antimicrobial properties and are selected from Ag, Au, Pt, Pd, Ir, Sn, Cu, Sb, Bi or Zn, or alloys or combinations thereof. More preferably, the metal nanoparticles have antimicrobial properties and comprise silver.

Alternatively, the metal nanoparticles may be selected so as to impart, for example, electrical or thermal conductivity, magnetism or improved fire retardancy to the extruded polymer. Examples of metal nanoparticles that impart magnetic properties are Co, Fe, Cu and NdFeB alloy.

In a preferred embodiment of the present invention, the metal nanoparticles comprise silver, the polymer matrix comprises alginate and fibres are produced by a spinning technique. Suitably, the silver is present in the alginate matrix in an amount between 0.1 and 15% w/w, more preferably between 0.1 and 2% w/w. In applications where a hydrophilic polymer is used, such as alginate, the amount of metal in the fibre is preferably kept low so that the water absorption properties of the fibre are not inhibited.

In another preferred embodiment, fibres are produced by a spinning technique and comprise polyacrylonitrile (PAN) and silver. Spun PAN/Ag fibres are particularly useful for producing antimicrobial fabrics.

Advantageously, the dope solution is de-aerated before extrusion, for example either under vacuum or by leaving the solution to stand in an inert atmosphere. Preferably, where a spinning technique is used, the polymer solution is also filtered prior to extrusion, so as to prevent blockage of the spinneret. The type of filtration system used depends on the spinneret hole diameter and the type and size of the particles that need to be removed from the solution. The filter system is preferably arranged behind the spinneret, and may be positioned in the spinneret holder. One preferred filter system comprises a wire mesh of size 150-500 micron, optionally arranged together with a thin, non-woven, or plain woven, fabric which is insoluble in the dope solution. Advantageously, an additional cartridge filter is positioned upstream of the spinneret.

Different types of spinnerets can be used for the production of polymer composite fibres, depending on the properties required in the final fibres; for example, geometric spinnerets can be used to obtain different cross-sectional shapes. For wet spinning, spinnerets with hole diameters less than 100 micron are preferable, more preferably between 60 and 90 micron. For dry spinning and dry-jet wet spinning, the holes are usually spaced out from each other to avoid the fluid filaments twinning during extrusion. Furthermore, the hole diameters are usually bigger than those used for wet spinning and range mostly above 100 micron.

For wet spinning, a coagulant containing a small amount of the solvent at or below ambient temperature is typically employed. For cellulose, for example, water or water containing a small amount of N-methylmorpholine-N-oxide (NMMO) at about 50 to 80° C. is typically employed as a coagulant. For alginate, aqueous Ca2+ is commonly used as the coagulant. For dry-jet wet spinning, the coagulant is the same as for wet spinning except that the bath could be at any temperature between 5 and 80° C. In all cases, the amount of NMMO in the coagulant is increased as extrusion progresses.

It will be appreciated that polymer composite fibres produced by the present invention, having metal nanoparticles incorporated throughout the body of the fibre, are superior to a woven or non-woven fibrous mass with the metal merely impregnated therein. In particular, the property exhibited by the fibres due to the metal is long-lived and not reduced by physical or chemical abrasion of the fibre etc. In the case of antimicrobial wound dressings comprising composite alginate/Ag fibres made according to the method of the present invention, antimicrobial activity is long lasting and deterioration of the alginate fibres by hydrolysis merely releases more of the antimicrobial metal.

According to a second aspect of the present invention, there are provided fibres comprising a polymer matrix having at least one metal incorporated therein, wherein the at least one metal is in the form of nanoparticles.

The present inventors have found that, by selecting certain combinations of polymer matrix and metal nanoparticles, composite fibres exhibiting a wide range of desirable properties can be obtained, for a variety of different applications; examples of fibres so-obtained are antimicrobial fibres, heat conducting fibres, electrically conductive fibres and magnetic fibres.

The metal nanoparticles are preferably distributed across the fibre cross section in a substantially uniform manner. In some applications, the concentration of metal particles distributed along the fibre length may be constant, but, alternatively, the concentration of metal particles along the fibre length may vary. In some instances, fibres according to the present invention may comprise one or more lengths of the base polymer in combination with one or more lengths of the polymer matrix incorporating metal nanoparticles. Thus, polymer fibres may be obtained that exhibit the required property either throughout their whole length or along part of their length.

There may be a degree of nanoparticle agglomeration in the polymer matrix, and, typically, nanoparticle agglomerates may be from 100 nm to 2 microns in size. For some applications, it may be preferable that agglomeration is controlled or even substantially avoided, so that the nanoparticles of even size are distributed throughout the polymer matrix.

In the case of hydrophilic fibres used in antimicrobial wound dressings and the like, particularly silver/alginate fibres, the gradual absorption of aqueous exudate into the polymer matrix leads to a gradual degradation of the matrix. By incorporating antimicrobial particles throughout the fibre according to the present invention, said particles are gradually released as the fibre absorbs fluid, thereby providing a controlled, slow release of the metal antimicrobial agent.

The fibres may have any diameter suitable for a given application, but typically the fibre diameter is less than 500 microns, more preferably less than 100 microns and most preferably 10 to 50 microns. Antimicrobial fibres for use in wound dressings, in particular, require fibre diameters in the above-mentioned ranges.

In some applications, the fibre may further comprise an outer protective sheath or coating.

In accordance with a third aspect of the present invention, there is provided a wound dressing comprising fibres according to the present invention. Preferably the wound dressing is a non-woven wound dressing.

In accordance with a fourth aspect of the present invention, there is provided a film comprising a polymer matrix having metal nanoparticles incorporated therein.

In accordance with a fifth aspect of the present invention, there is provided a polymer composite comprising a polymer matrix having metal nanoparticles incorporated therein.

A preferred embodiment of the present invention will now be described, with reference to the accompanying drawings in which:

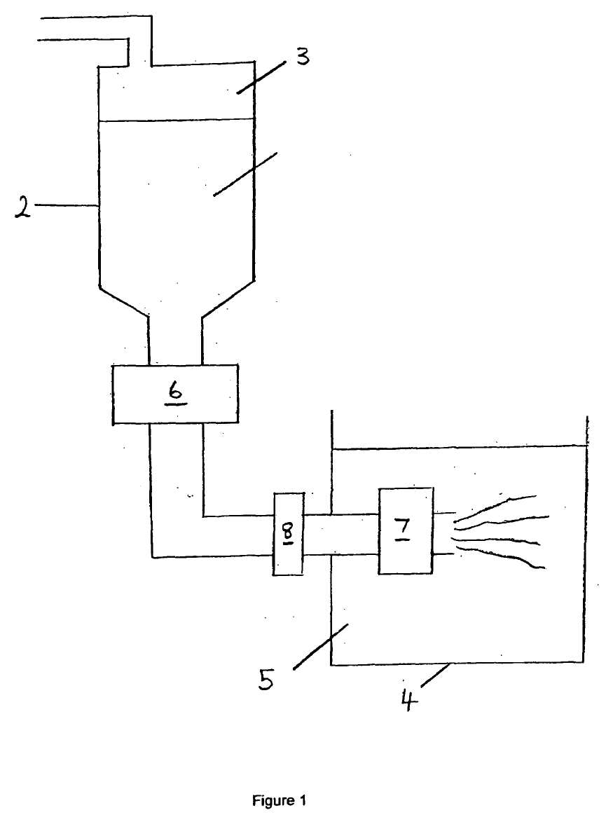

FIG. 1 is a schematic sectional view of a preferred apparatus for wet-spinning composite fibres according to the present invention;

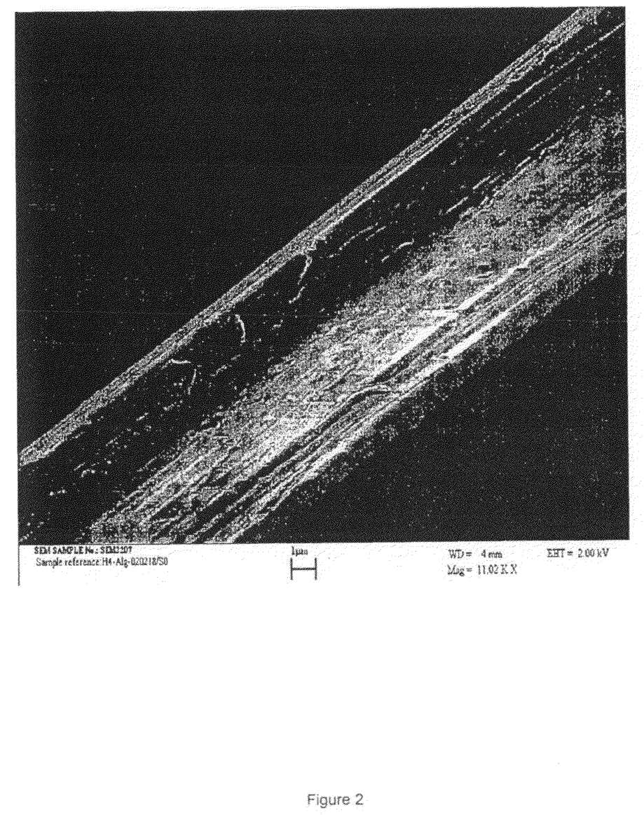

FIG. 2 shows a SEM spectrum of the longitudinal outer surface of a prior art alginate fibre;

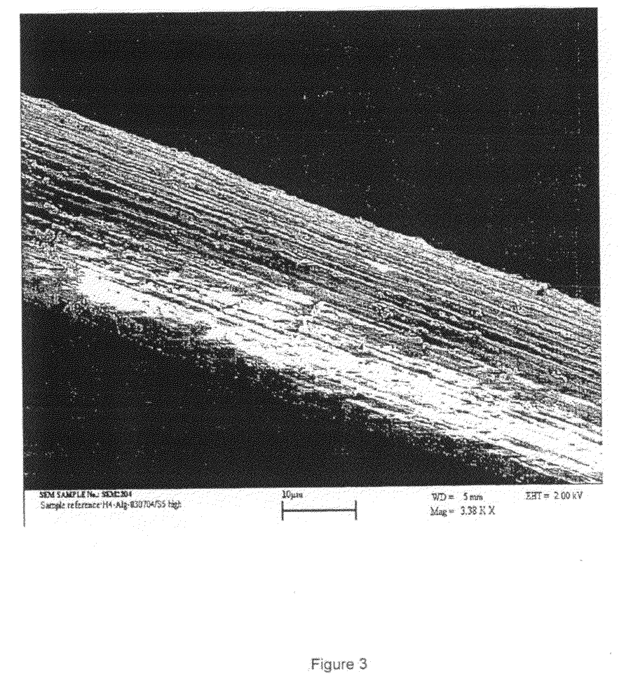

FIG. 3 shows a SEM spectrum of the longitudinal outer surface of an alginate fibre incorporating 5% w/w silver nanoparticles according to the present invention;

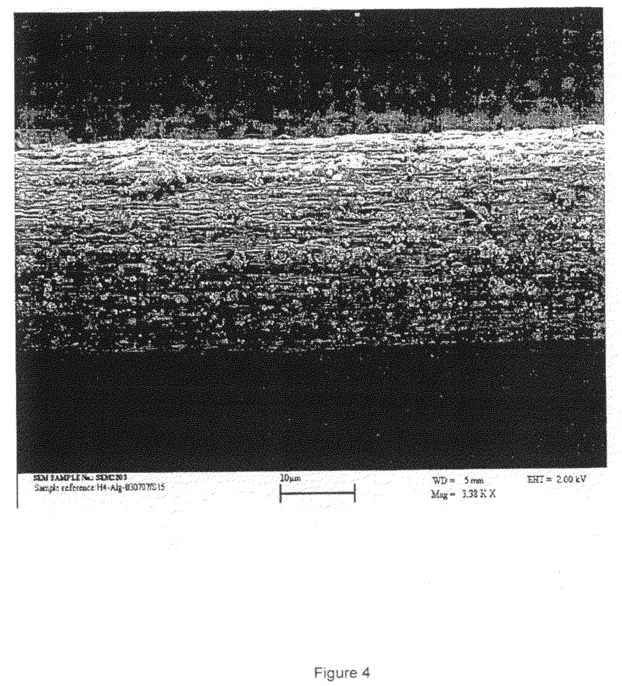

FIG. 4 shows a SEM spectrum of the longitudinal outer surface of an alginate fibre incorporating 15% w/w silver nanoparticles according to the present invention; and

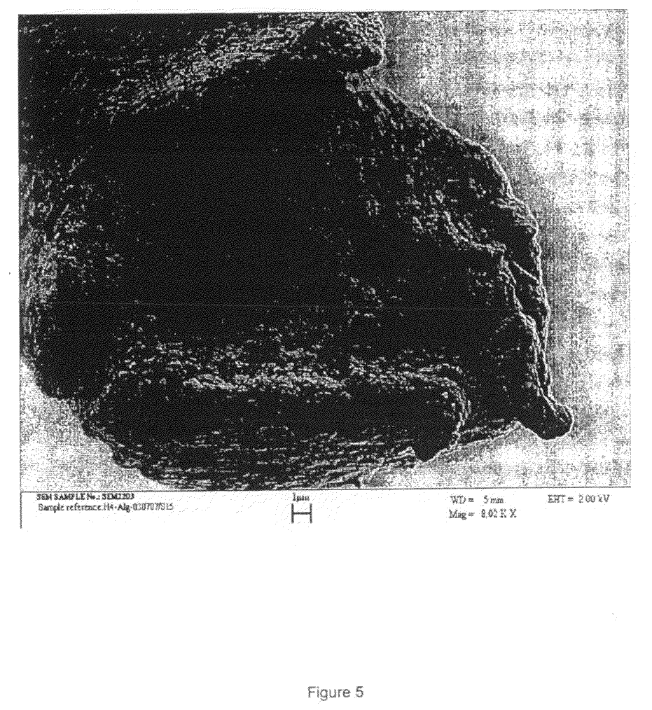

FIG. 5 shows a SEM spectrum of a cross-section of an alginate fibre incorporating 15% w/w silver nanoparticles according to the present invention.

Fibres comprising an alginate matrix having silver nanoparticles incorporated therein can be used in biomedical applications such as wound dressings.

Alginate is a linear polysaccharide made up of two uronic acid monomers, mannuronic acid (M) and guluronic acid (G). The ratio of the two monomers (the ‘M:G ratio’) and their arrangement in the polymer structure vary from source to source and, to a large extent, control the chemical and physical properties of the alginate. For example, more guluronic segments in the alginate provide a stronger gel or fibre. The two monomers are connected in blocks of M-M, G-G or M-G sequences in the polymer structure. Preferably, the alginate fibres are formed either from High-G alginate having a viscosity of 1% solution of 50-100 mPa or High-M alginate having a viscosity of 1% solution of 40-80 mPa.

Referring to FIG. 1, alginate/silver fibres are produced by first dissolving 5-7% w/w of the sodium salt of the polymer in water, and then mixing 0.1-15% w/w of a silver nanopowder into the solution so as to produce a dope solution 1. The dope solution is held in a container 2 provided with an inert atmosphere 3, and then the dope is extruded into a coagulating bath 4 containing a coagulant 5, by means of a pump 6 and a spinneret head 7 completely immersed in the coagulant. The dope is filtered via a filter 8 positioned behind the spinneret.

FIG. 2 show a SEM spectrum of the outer surface of a prior art alginate fibre, without metal nanoparticles incorporated therein: FIG. 3 shows a SEM image of a similar alginate fibre according to the present invention, comprising 5% w/w silver nanoparticles; silver nanoparticles and nanoparticle agglomerates can be seen on the outer surface.

FIG. 4 shows an alginate fibre according to the present invention having 15% w/w silver incorporated in the fibre. A higher concentration of particles and particles agglomerates can been seen compared with FIG. 3.

FIG. 5 is a SEM image of a cross-section of the fibre shown in FIG. 4. It can be seen that the silver nanoparticles and agglomerates thereof are distributed throughout the fibre cross-section, and, therefore, that the fibre is a composite material comprising silver nanoparticles incorporated in an alginate matrix.

The present invention provides fibres and other products that may be used alone or in combination, and applications are not limited to those described herein. It will be clear to the skilled person that the method of the present invention can be used to provide polymeric materials for use in a wide variety of bio-medical applications and textile applications, including, for example, mould-resistant products such as tent fabrics, tropical clothing, leisure wear and sportswear.

Anti-microbial fibres may also be used in water filtration systems where, currently, a nylon base fibre is used.

The following Examples further describe the invention

### EXAMPLE 1

### Alginate/Silver Fibre

Composite fibres comprising silver nanoparticles incorporated into an alginate polymer were made as follows:

A 500 g (5% w/w) high G alginate (supplied by ISP Alginates (UK) Ltd) was dissolved in 470 g of water using a high shear mixer. The dope solution thus formed was stirred for 15-20 minutes and then 5 g (1% w/w) nanometric silver powder having a particle size between about 20 and 100 nm (particle size distribution d90<70 nm, d50<50 nm and d10<40 nm) was gradually added to the dope solution whilst stirring continuously for further 30-45 minutes. (The heat of mixing increased to about 60° C.) The solution thus prepared was vacuum de-aerated and then wet extruded through a filter system (300 wire mesh, non-woven fabric and plain woven fabric) and a spinneret into an aqueous calcium chloride bath at ambient temperature. After drawing in a hot water bath, the fibres were washed in acetone and finally dried at room temperature.

The alginate/silver fibres so-obtained ranged from a very pale brown colour to a dark brown/black colour. The fibres exhibited both bacteriostatic and bactericidal activity against Staphylococcus aureus and Klebsiella pneumoniae, and also demonstrated a good antifungal effect against Trichophyton mentagrophytes.

### EXAMPLE 2

### Cellulose/Silver Fibre

35 g cellulose wood pulp (Acordis) having a degree of polymerisation similar to pulp used for the manufacture of Lyocell fibres was broken into small pieces and mixed with N-methylmorpholine-N-oxide (NMMO) at 100-120° C. The mixture was stirred, using a high shear mixer, for 30-60 minutes to form a slightly coloured 10% w/w polymer solution, or dope. The solution was allowed to cool down to 90-100° C. and then 17.5 g (5% w/w) of nanometric silver powder of the same size used in Example 1 was added gradually into the dope. The dope was stirred continuously, typically for 5 to 15 minutes, so that a homogeneous mixture was obtained. It was found that the process was highly exothermic and the temperature had to be maintained well below 120° C. The final dope (350 g) contained 15% w/w total solids.

The cellulose-silver fibre was prepared by extruding the hot mixture, under nitrogen pressure (0.3-0.4 MPa), through a filter system (300 wire mesh, fine woven polyester) and a heated spinneret with 35 holes of size 90 micron, immersed in a water coagulant at 55-60° C. The newly formed filament was then drawn in a hot water bath at 75-80° C., wound unto a roller immersed in a dehydrating agent such acetone and dried on the drum at room temperature, or in an oven at 50-80° C.

The cellulose/silver fibres ranged from a very pale brown colour to a dark brown/black colour. The fibres exhibited bacteriostatic and bactericidal activity against Staphylococcus aureus and Klebsiella pneumoniae, and also demonstrated a good antifungal effect against Trichophyton mentagrophytes.

### EXAMPLE 3

### Cellulose/Copper Fibre

24.5 g cellulose wood pulp (Acordis) was cut into small pieces and mixed with 322 g of NMMO at 100-110° C. The mixture was stirred, using a high shear mixer, for 30-60 minutes to form a 7% w/w dope. The dope solution was allowed to cool down to 100° C. and then 3.5 g (1% w/w) of nanometric copper powder with a nominal primary particle size, as determined from specific surface area measurements, of 100 nm was added gradually into the dope whilst stirring continuously, until a homogeneous mixture was obtained. The process was highly exothermic and the temperature had to be maintained well below 120° C. The final dope contained 8% w/w total solids. The cellulose-copper fibre was prepared by extruding the hot mixture (70-100° C.) under nitrogen pressure (0.3-0.4 MPa) through a filter system and a heated spinneret (80 micron/500 holes) immersed in a water coagulant at 55-65° C. The filaments thus-formed were drawn in a hot water bath at 75-80° C., collected in a tray containing acetone and dries at room temperature. Alternatively, the filaments were wound onto a roller immersed in acetone before finally dried at room temperature.

The cellulose/copper fibres exhibited antimicrobial and magnetic properties.

### EXAMPLE 4

### Polyacrylonitride/Copper Fibre

Examples 4 and 5 demonstrate the versatility of the process, which is applicable to all spun polymer systems (synthetic, regenerated or natural), provided the solution or melt does not attack the metals.

The dope was prepared by soaking 96 g polyacrylonitrile (PAN) fibre (20% w/w) in 383 g N,N-dimethylacetamide (DMAc) at about 60° C. until fully dissolved. The solution was then stirred at 60-70° C. for 5 minutes with a high shear mixer before gradually adding 2.4 g copper powder (0.5% w/w, nominal primary particle size 100 nm) and stirring continuously so as to obtain a homogeneous solution. The 40° C. dope thus obtained was then spun into a water/DMAc (30% v/v) bath at room temperature, drawn in a hot water bath and, after winding onto a roller, was left to dry at room temperature.

### EXAMPLE 5

### Polyacrylonitrile/Silver Fibre

The dope was prepared at room temperature using a high shear mixer to dissolve 48 g (12%, w/w) PAN fibre in 350 g aqueous sodium thiocyanate solution containing 200 g (50% w/w) of the thiocyanate. About 2 g of silver powder corresponding to 0.5% w/w of the dope was then gradually added to the dope whilst stirring continuously until the powder was homogeneously dispersed. The dope thus obtained was vacuum de-aerated, spun into 11% aqueous sodium thiocyanate solution, drawn in a hot water bath, wound onto a roller and dried in an oven between 50 and 60° C. for about 18 hours.

Polyacrylonitrile/silver fibres exhibited antibacterial activity against Staphylococcus aureus and Klebsiella pneumoniae, and antifungal activity against Trichophyton mentagrophytes

1. A method of producing polymer composite fibres comprising a polymer matrix having metal nanoparticles incorporated therein, said method comprising the steps of:
(i) mixing metal nanoparticles with a polymer dope; and
(ii) solidifying the polymer composite from the dope by a fibre extrusion process.

2. A method according to claim 1, wherein the dope is stirred vigorously so as to produce a homogeneous mixture.

3. A method according to claim 2, wherein a high shear mixer is used to stir the dope.

4. A method according to claim 1, wherein the metal nanoparticles are added directly to the polymer dope as a powder.

5. A method according to claim 1, wherein the metal nanoparticles comprise one or more transition metals.

6. (canceled)

7. (canceled)

8. A method according to claim 7, wherein the fibres are extruded by a spinning technique.

9. A method according to claim 8, wherein the fibres are extruded by a wet spinning technique.

10. A method according to claim 1, wherein the polymer dope comprises a linear polymeric material having fibre forming characteristics.

11. A method according to claim 1, wherein the metal nanoparticles have antimicrobial properties.

12. A method according to claim 11, wherein the nanoparticles comprise silver.

13. A method according to claim 1, wherein the polymer matrix comprises alginate.

14. A method according to claim 1, wherein the polymer matrix comprises polyacrylonitrile.

15. A method according to any preceding claim 1, wherein the metal nanoparticles have a size less than 500 nm.

16. A method according to claim 15, wherein the metal nanoparticles have a size less than 100 nm.

17. A method according to claim 16, wherein the metal nanoparticles have a size in the range 20 to 100 nm.

18. (canceled)

19. (canceled)

20. (canceled)

21. Fibres comprising a polymer matrix having at least one metal incorporated therein, wherein the at least one metal is in the form of nanoparticles.

22. Fibres according to claim 21, wherein the nanoparticles are distributed in a substantially uniform manner across the fibre cross section.

23. Fibres according to claim 21, wherein the metal nanoparticles have a size less than 500 nm.

24. Fibres according to claim 23, wherein the metal nanoparticles have a size less than 100 nm.

25. Fibres according to claim 24, wherein the metal nanoparticles have a size in the range 20 to 100 nm.

26. Fibres according to claim 21, wherein the metal nanoparticles have antimicrobial properties.

27. Fibres according to claim 26, wherein the metal nanoparticles comprise Ag, Au, Pt, Pd, Ir, Sn, Cu, Sb, Bi, or Zn, or any combination thereof.

28. Fibres according claim 27, wherein the metal nanoparticles comprise Ag.

29. Fibres according to claim 21 having a diameter of less than 500 microns.

30. Fibres according to claim 29 having a diameter of less than 100 microns.

31. Fibres according to claim 30 having a diameter of 10 to 50 microns.

32. Fibres according to claim 21, wherein the polymer matrix comprises a synthetic polymer, a natural polymer or any combination thereof.

33. Fibres according to claim 32, wherein said natural polymer comprises alginate.

34. Fibres according claim 33, wherein the polymer matrix comprises alginate and Ag is present in the polymer matrix in an amount between 0.1 and 15% w/w, and preferably in an amount between 0.1 and 2% w/w.

35. Fibres according to claim 32, wherein said synthetic polymer comprises polyacrylonitrile.

36. Fibres according claim 35, wherein the polymer matrix comprises polyacrylonitrile and Ag is present in the polymer matrix in an amount between 0.05 and 2% w/w.

37. A wound dressing comprising fibres according to claim 33.

38. A woven or non-woven fibrous article comprising fibres according to claim 21, particularly a fabric comprising said fibres.

39-42. (canceled)
